(19) United States
(12) Patent Application Publication
(10) Pub. No.: US 2025/0000388 A1
(43) Pub. Date: Jan. 2, 2025

(54) DETECTION SYSTEM, DETECTION METHOD, AND A NON-TRANSITORY COMPUTER-READABLE STORAGE MEDIUM

(71) Applicant: PANASONIC INTELLECTUAL PROPERTY MANAGEMENT CO., LTD., Osaka (JP)

(72) Inventors: Makoto YASUGI, Tokyo (JP); Takeo UETA, Kanagawa (JP); Toru OKADA, Kanagawa (JP); Hiroshi NOGUCHI, Kanagawa (JP); Kosuke ONO, Kanagawa (JP)

(73) Assignee: PANASONIC INTELLECTUAL PROPERTY MANAGEMENT CO., LTD., Osaka (JP)

(21) Appl. No.: 18/883,735

(22) Filed: Sep. 12, 2024

Related U.S. Application Data
(63) Continuation of application No. PCT/JP2023/007167, filed on Feb. 27, 2023.

(30) Foreign Application Priority Data
Mar. 18, 2022 (JP) 2022-044144

(51) Int. Cl. A61B 5/11; A61B 5/00; A61B 5/05

(52) U.S. Cl. CPC A61B 5/1118; A61B 5/05; A61B 5/743

(57) ABSTRACT

A detection system includes at least one radar type sensor installed in a predetermined area, a biological information detection device configured to detect biological information of a person in the area based on sensor data output from the sensor, and a terminal device configured to generate and display support information for supporting installation of the sensor using the detected biological information.

### CROSS-REFERENCE TO RELATED APPLICATIONS

This is a continuation of International Application No. PCT/JP2023/007167 filed on Feb. 27, 2023, and claims priority from Japanese Patent Application No. 2022-044144 filed on Mar. 18, 2022, the entire content of which is incorporated herein by reference.

### TECHNICAL FIELD

The present disclosure relates to a detection system, a detection method, and a non-transitory computer-readable storage medium.

### BACKGROUND ART

In the related art, a vital sign sensor is known that transmits a modulated wave in a microwave band or a millimeter wave band, compares the transmitted wave with a received wave reflected from an object, and detects a minute variation such as respiration, heartbeat or the like of a living body (for example, Patent Literature 2).

### CITATION LIST

### Patent Literature

Patent Literature 1: WO2021/039601
Patent Literature 2: JP2019-152441A
Patent Literature 3: JP2016-138796A

### Technical Problem

According to the related art, it is possible to detect an object with high accuracy and acquire biological information such as respiration and heartbeat while maintaining privacy. However, in the technique in the related art, an installation adjustment of the vital sign sensor has to depend on the understanding based on experience of a worker as a preparation before the acquisition of the biological information, and it is desired to construct an appropriate acquisition environment of the biological information while receiving guidance of the installation adjustment. The technique in the related art does not consider such demands at all.

An object of the present disclosure is to provide a technique for supporting installation of a sensor.

### SUMMARY OF INVENTION

A detection system according to an aspect of the present disclosure includes: at least one radar type sensor installed in a predetermined area; a biological information detection device configured to detect biological information of a person in the area based on sensor data output from the sensor; and a terminal device configured to generate and display support information for supporting the installation of the sensor using the detected biological information.

A detection method according to an aspect of the present disclosure includes: detecting biological information of a person in a predetermined area based on sensor data output from at least one radar type sensor installed in the area; and generating and displaying support information for supporting the installation of the sensor using the detected biological information.

A non-transitory computer-readable storage medium according to an aspect of the present disclosure has a computer program stored thereon and readable by a computer, the computer program, when executed by the computer, causing the computer to execute: detecting biological information of a person in a predetermined area based on sensor data output from at least one radar type sensor installed in the area; and generating and displaying support information for supporting the installation of the sensor using the detected biological information.

These comprehensive or specific aspects may be implemented by a system, a device, a method, an integrated circuit, a computer program, or a recording medium, or any combination of the system, the device, the method, the integrated circuit, the computer program, and the recording medium.

According to the present disclosure, a technique for supporting installation of a sensor can be provided.

### BRIEF DESCRIPTION OF DRAWINGS

FIG. 1 is a block diagram showing a configuration example of a detection system according to Embodiment 1;

FIG. 2 is a diagram showing a configuration example of sensor data according to Embodiment 1;

FIG. 3 is a diagram showing a configuration example of biological information according to Embodiment 1;

FIG. 4 is a flowchart showing an example of setting processing of biological degree determination information and display setting information by the detection system according to Embodiment 1;

FIG. 5 is a flowchart showing an example of monitoring processing of a monitoring area by the detection system according to Embodiment 1;

FIG. 6 is a diagram showing a selection screen of a usage scene “watching” according to Embodiment 1;

FIG. 7 is a diagram showing an example of a setting screen of the biological degree determination information for the usage scene “watching” according to Embodiment 1;

FIG. 8 is a diagram showing a setting screen of the display setting information for the usage scene “watching” according to Embodiment 1;

FIG. 9 is a diagram showing a setting screen of biological degree correction setting information for the usage scene “watching” according to Embodiment 1;

FIG. 10 is a diagram showing an example of a monitoring screen when a person is sleeping in a bedroom according to Embodiment 1;

FIG. 11 is a diagram showing an example of a monitoring screen when a person is watching television in a living room according to Embodiment 1;

FIG. 12 is a diagram showing an example of a monitoring screen when the person is in an abnormal state in a bathroom according to Embodiment 1;

FIG. 13 is a diagram showing a selection screen of a usage scene “abnormality detection” according to Embodiment 1;

FIG. 14 is a view showing an example of a setting screen of the biological degree determination information for the usage scene “abnormality detection” according to Embodiment 1;

FIG. 15 is a diagram showing a setting screen of the display setting information for the usage scene “abnormality detection” according to Embodiment 1;

FIG. 16 is a view showing a setting screen of the biological degree correction setting information for the usage scene “abnormality detection” according to Embodiment 1;

FIG. 17 is a diagram showing an example of a monitoring screen when two persons are in an office according to Embodiment 1;

FIG. 18 is a diagram showing an example of a monitoring screen when there is a person in an abnormal state in an office according to Embodiment 1;

FIG. 19 is a diagram showing an example of a selection screen of a usage scene “sensor installation support” according to Embodiment 2;

FIG. 20 is a diagram showing an example of a setting screen of sensor installation information according to Embodiment 2;

FIG. 21 is a diagram showing an example of a sensor installation confirmation support screen according to Embodiment 2;

FIG. 22 is a diagram showing an example of a sensor range confirmation support screen according to Embodiment 2;

FIG. 23 is a diagram showing an example of a result confirmation screen according to Embodiment 2; and

FIG. 24 is a diagram showing a hardware configuration of a computer that implements functions of each device according to the present disclosure by a computer program.

### DESCRIPTION OF EMBODIMENTS

Hereinafter, embodiments of the present disclosure will be described in detail with reference to the drawings as appropriate. However, unnecessarily detailed description may be omitted. For example, detailed description of already well-known matters and redundant description of substantially the same configuration may be omitted. This is to avoid unnecessary redundancy of the following description and to facilitate understanding of those skilled in the art. It should be noted that the accompanying drawings and the following description are provided for those skilled in the art to sufficiently understand the present disclosure, and are not intended to limit the subject matter described in claims.

### Embodiment 1

### <System Configuration>

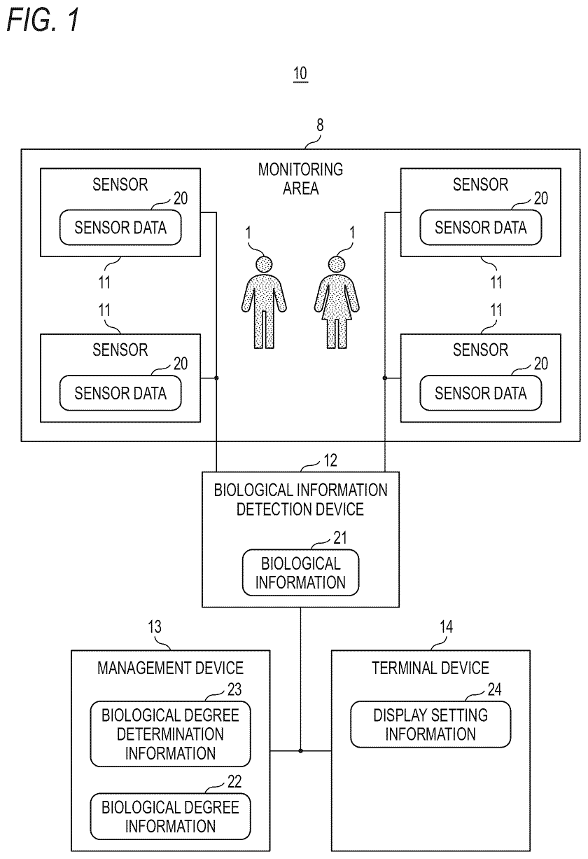

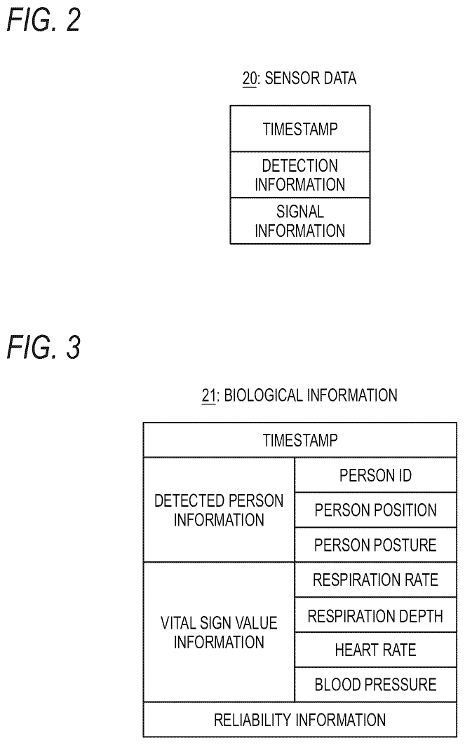

FIG. 1 is a block diagram showing a configuration example of a detection system 10 according to Embodiment 1. FIG. 2 is a diagram showing a configuration example of sensor data 20 according to Embodiment 1. FIG. 3 is a diagram showing a configuration example of biological information 21 according to Embodiment 1.

The detection system 10 includes a sensor 11, a biological information detection device 12, a management device 13, and a terminal device 14. The sensor 11, the biological information detection device 12, the management device 13, and the terminal device 14 may transmit and receive data or information through a predetermined communication network. The communication network may be wired or wireless.

A plurality of the sensors 11 may be disposed so as to monitor an entire monitoring area 8.

The sensor 11 is a radar type sensor, radiates a radio wave in a predetermined frequency band, and receives reflected waves reflected from an object (for example, a person 1) present in a sensing range of the sensor 11. A sensor 11 compares the radiated radio wave with the received reflected wave to generate the sensor data 20 shown in FIG. 2. The sensor 11 may be a millimeter wave radar type sensor. Examples of the millimeter wave radar type include a frequency modulated continuous wave (FMCW) type and a stepped interrupted continuous wave (ICW) type.

In the sensor data 20, as shown in FIG. 2, a timestamp, detection information, and signal information are associated with each other.

The timestamp is information indicating a timing at which the sensor data 20 is obtained. Examples of the timestamp include time information or a frame number.

The detection information includes, for example, information indicating a position (hereinafter, referred to as a reflection point) in a three-dimensional space where a reflected wave having a predetermined intensity or more is obtained, information indicating a distance from the sensor 11 to the reflection point, and information indicating a reflection intensity at the reflection point.

The signal information includes, for example, IQ data for each reception antenna.

That is, the sensor data 20 is data indicating a position, a shape, and a temporal change of an object present in the sensing range in the three-dimensional space, and does not have a level of resolution of video data, and thus each individual cannot be specified, and data acquisition is possible in a state in which privacy of a person is protected by the sensor 11.

The sensor 11 transmits the generated sensor data 20 to the management device 13 and the terminal device 14.

The biological information detection device 12 generates biological information 21 shown in FIG. 3 based on the sensor data 20 received from each sensor 11.

In the biological information 21, as shown in FIG. 3, the timestamp, detected person information, vital sign value information, and reliability are associated with each other.

The timestamp is information indicating a timing at which the biological information 21 is generated. Examples of the timestamp include time information. The management device 13 sets the timing when the biological information 21 is generated as the timestamp.

The detected person information is information related to the detected person 1. The detected person information includes a person ID, a person position, and a person posture. The person ID is an ID for identifying the detected person 1. The person position indicates the position of the person indicated by the person ID in the three-dimensional space in the monitoring area 8. The person posture indicates the posture of the person 1 indicated by the person ID. The biological information detection device 12 specifies the person position and the person posture based on the detection information of the sensor data 20.

The vital sign value information is information related to a vital sign value that is a detected activity state of the person 1. Examples of the vital sign value include a respiration rate, a respiration depth, a heart rate, and blood pressure. The biological information detection device 12 calculates the vital sign value (for example, the respiration rate, the respiration depth, the heart rate, the blood pressure, or the like) of the detected person 1 based on a temporal change in the detection information of the sensor data 20.

Reliability information is information indicating reliability (likelihood) of the vital sign value information. The biological information detection device 12 calculates the reliability of the calculated vital sign value information based on the detection information and the signal information of sensor data 20. The reliability information can be generated based on a noise level ratio of a radar signal which is a calculation source of the vital sign value information or a comparison result of reliability information of a plurality of different vital sign value information candidates.

The biological information detection device 12 transmits the generated biological information 21 to the management device 13 and the terminal device 14.

The management device 13 generates biological degree information 22 of each person 1 based on the biological information 21 received from the biological information detection device 12. The biological degree information 22 is information indicating a degree (level) of the vital sign value which is the activity state of person 1. At this time, the management device 13 generates the biological degree information 22 based on biological degree determination information 23. The biological degree determination information 23 includes information for determining the degree from the vital sign value. A user can set the biological degree determination information 23 by operating the management device 13. Details of the biological degree determination information will be described later.

The management device 13 transmits the generated biological degree information 22 and biological degree determination information 23 to the terminal device 14.

The terminal device 14 estimates an action or a state of the person 1 in the monitoring area 8 based on the biological information 21 received from the biological information detection device 12 and the biological degree information 22 and the biological degree determination information 23 received from the management device 13, and displays the action or the state on a monitoring screen. At this time, the terminal device 14 determines a display mode of the action or the state of the person 1 based on display setting information 24. The display setting information 24 is information including setting of the display mode of the action or the state of the person. The user can set the display setting information 24 by operating the terminal device 14. Details of the display setting information 24 will be described later.

As described above, the detection system 10 detects the biological state of the person 1 using the radar type sensor 11 that senses the person using the radio wave. Accordingly, the biological state of the person can be detected while the privacy of the person 1 is protected.

### <Setting Processing Flow>

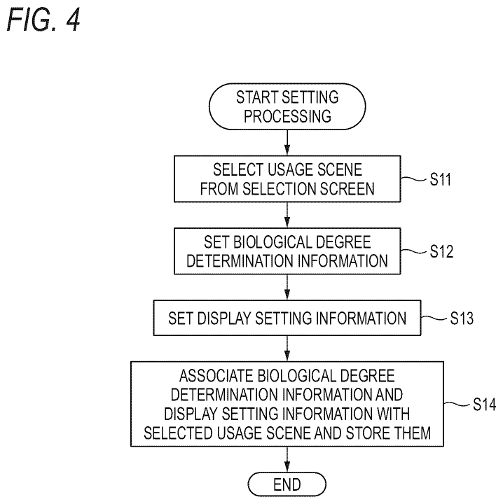

FIG. 4 is a flowchart showing an example of setting processing of the biological degree determination information 23 and the display setting information 24 by the detection system 10 according to Embodiment 1.

The management device 13 displays a selection screen of a usage scene. A specific example of the selection screen will be described later. The user selects one usage scene from the selection screen (S11).

The management device 13 reads and displays default biological degree determination information 23 for the usage scene selected in step S11. The user sets the displayed biological degree determination information 23 (S12). A method of setting the biological degree determination information 23 will be described in detail later.

The terminal device 14 displays the default display setting information 24 for the usage scene selected in step S11. The user sets the displayed display setting information 24 (S13). A method of setting the display setting information 24 will be described in detail later.

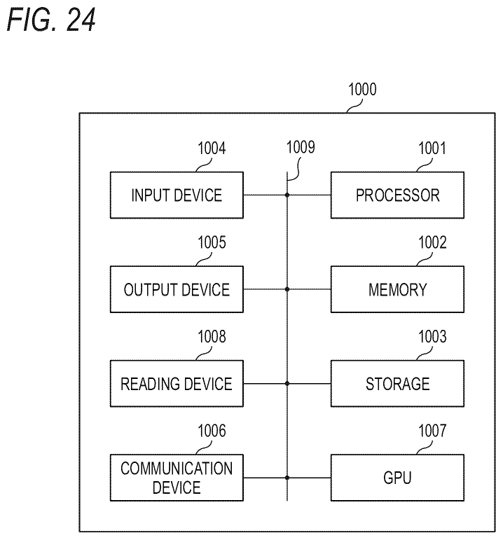

The management device 13 associates the biological degree determination information 23 set in step S12 with the usage scene selected in step S11 and stores them in a storage 1003 (see FIG. 24). The terminal device 14 associates the display setting information 24 set in step S13 with the usage scene selected in step S11 and stores them in the storage 1003 (S14).

According to the above processing, the biological degree determination information 23 and the display setting information 24 appropriate for each usage scene are associated and stored in the storage 1003.

### <Monitoring Processing Flow>

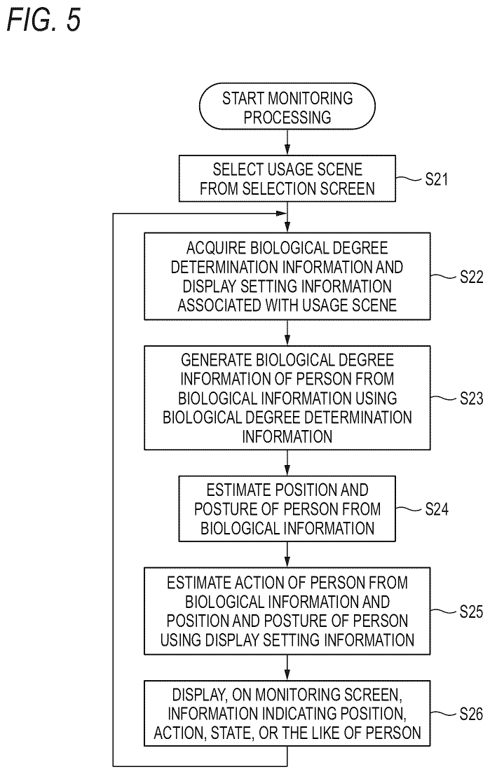

FIG. 5 is a flowchart showing an example of monitoring processing of the monitoring area 8 by the detection system 10 according to Embodiment 1.

The terminal device 14 displays a selection screen of a usage scene. The user selects one usage scene from the selection screen (S21).

The management device 13 acquires the biological degree determination information 23 associated with the usage scene selected in step S21 from the storage 1003. The terminal device 14 acquires the display setting information 24 associated with the usage scene selected in step S21 from the storage 1003 (S22). Accordingly, the detection system 10 can perform the monitoring processing using the biological degree determination information 23 and the display setting information 24 appropriately set for the usage scene.

The management device 13 generates the biological degree information 22 from the biological information 21 received from the biological information detection device 12 using the biological degree determination information 23 (S23). The management device 13 transmits the generated biological degree information 22 to the terminal device 14.

The terminal device 14 specifies the position and the posture of the person 1 from the biological information 21 received from the biological information detection device 12 (S24).

The terminal device 14 estimates the action or state of the person from the position and the posture of the person and the biological degree information 22 using the display setting information 24 (S25).

The terminal device 14 displays information indicating the estimated position, action, state, or the like of the person on the monitoring screen 400 (S26). Then, the detection system 10 returns the processing to step S22.

Through the above processing, the user can intuitively monitor the position, action, or state (for example, an abnormal state related to a living body of the person 1) of the person 1 in the monitoring area 8 by viewing the monitoring screen.

### <Usage Scene: Watching>

Next, watching of an older person or the like, which is an example of a usage scene of the detection system 10, will be described.

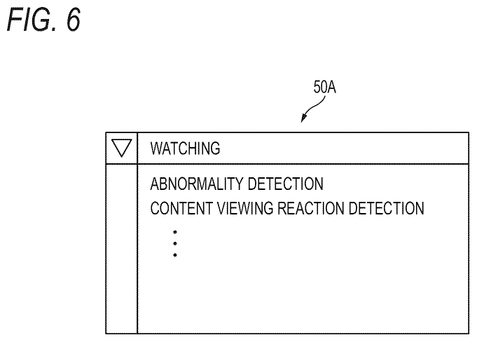

FIG. 6 is a diagram showing a selection screen 50A of the usage scene “watching” according to Embodiment 1.

In step S11 of FIG. 4, the management device 13 displays the selection screen of the usage scene shown in FIG. 6. When the user selects “watching” as the usage scene, the management device 13 displays a setting screen 100A of the biological degree determination information 23 for the usage scene “watching” to be described below.

### <Setting Screen for Watching>

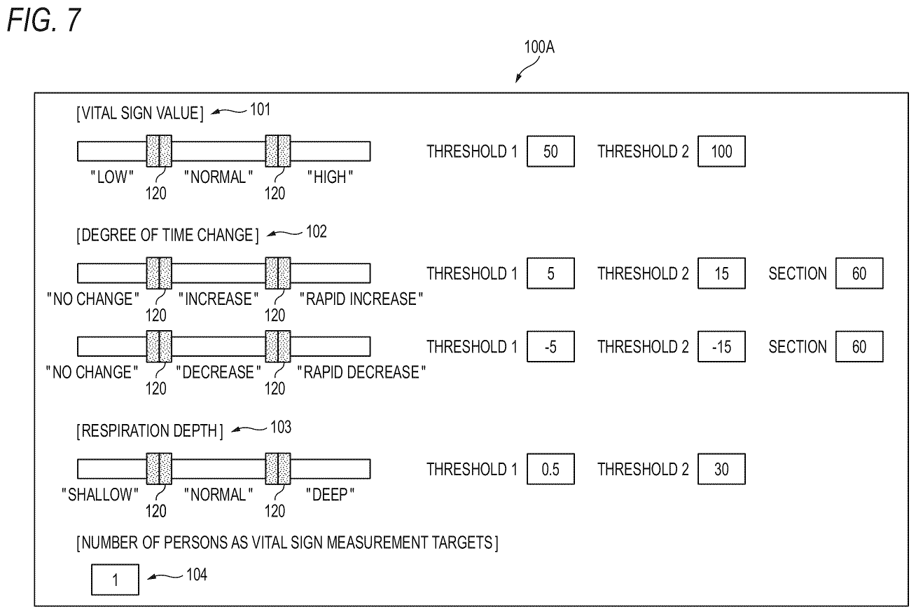

FIG. 7 is a diagram showing an example of the setting screen 100A of the biological degree determination information 23 for the usage scene “watching” according to Embodiment 1.

As shown in FIG. 7, the setting screen 100A of the biological degree determination information 23 for the usage scene “watching” includes a threshold setting region 101 of the vital sign value, a threshold setting region 102 of a degree of time change of the vital sign value, a threshold setting region 103 of the respiration depth, and a number-of-person-to-be-measured setting region 104.

The user adjusts threshold bars 120 included in the threshold setting region 101 of the vital sign value to set thresholds for determining a biological degree related to the height of the vital sign value from the vital sign value. The biological degree related to the height of the vital sign value is referred to as a vital sign degree. For example, when three levels of “low”, “normal”, and “high” are set as the vital sign degree, as shown in FIG. 7, the user sets a threshold 1 for distinguishing between “low” and “normal” and a threshold 2 for distinguishing between “normal” and “high”. For example, the user may set the thresholds to be low (that is, stricter) for the older person who is a watching target. The set threshold 1 and threshold 2 may be displayed as numerical values. The vital sign degree is not limited to having three levels, and may have two levels or four levels or more. A name of each level of the vital sign degree may be changeable by the user. Further, each level of the vital sign degree may be read as a vital sign height level.

The user adjusts the threshold bars 120 included in the threshold setting region 102 of the degree of time change of the vital sign value to set thresholds for determining the biological degree related to the degree of time change of the vital sign value from the degree of time change of the vital sign value. The biological degree related to the degree of time change of the vital sign value includes a degree in an increase direction level and a degree in a decrease direction. The degree in the increase direction is referred to as a degree of a vital sign increase change. The degree in the decrease direction is referred to as a degree of a vital sign decrease change. For example, when three levels of “no change”, “increase”, and “rapid increase” are set as the degree of the vital sign increase change, as shown in FIG. 7, a threshold 1 for distinguishing between “no change” and “increase” and a threshold 2 for distinguishing between “increase” and “rapid increase” are set. For example, the user may set the thresholds to be lower (that is, stricter) for the older person who is the watching target. The set threshold 1 and threshold 2 may be displayed as numerical values. Each level of the degree of the vital sign increase change may be read as a vital sign increase change level. For example, when three levels of “no change”, “decrease”, and “rapid decrease” are set as the degree of the vital sign decrease change, as shown in FIG. 7, the threshold 1 for distinguishing between “no change” and “decrease” and the threshold 2 for distinguishing between “decrease” and “rapid decrease” are set. For example, the user may set the thresholds to be lower (that is, stricter) for the older person who is the watching target. The set threshold 1 and threshold 2 may be displayed as numerical values. Each level of the degree of the vital sign decrease change may be read as a vital sign decrease change level.

The user adjusts the threshold bars 120 included in the threshold setting region 103 of the respiration depth to set thresholds for determining the biological degree related to the respiration depth of the vital sign value from the respiration depth of the vital sign value. The biological degree related to the respiration depth of the vital sign value is referred to as a degree of the respiration depth. For example, when three levels of “shallow”, “normal”, and “deep” are set as the degree of the respiration depth, as shown in FIG. 7, the user sets the threshold 1 for distinguishing between “shallow” and “normal” and the threshold 2 for distinguishing between “normal” and “deep”. For example, the user may set the thresholds to be lower (that is, stricter) for the older person who is the watching target. The set threshold 1 and threshold 2 may be displayed as numerical values. Each level of the degree of the respiration depth may be read as a respiration depth level.

The user sets a maximum number of persons as measurement targets in the number-of-person-to-be-measured setting region 104. Hereinafter, the set maximum number of persons is referred to as a maximum number of persons to be measured. For example, the user may set “one person” when the older person who is the watching target lives alone in a room which is the monitoring area. Typically, the reliability of the biological information is improved by reducing the maximum number of persons to be measured.

The management device 13 generates the biological degree determination information 23 including each threshold and the maximum number of persons to be measured set by the user, and stores the biological degree determination information 23 in a storage 1003 in association with the usage scene “watching”. Accordingly, when the usage scene “watching” is selected in the monitoring processing shown in FIG. 5, the management device 13 can use the biological degree determination information 23 appropriately set for the usage scene “watching”.

### <Display Setting Information for Watching>

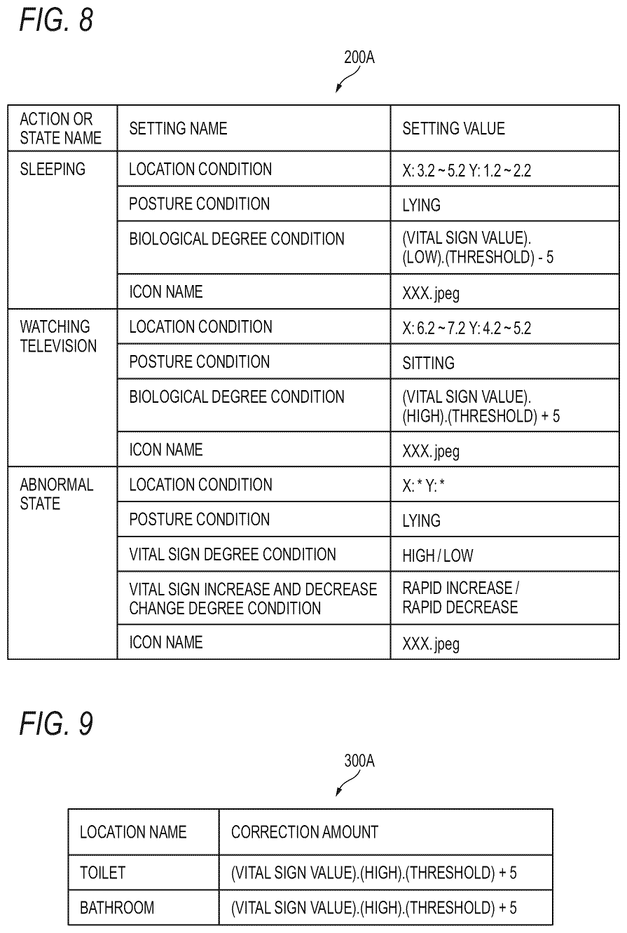

FIG. 8 is a diagram showing a setting screen 200A of the display setting information 24 for the usage scene “watching” according to Embodiment 1.

As shown in FIG. 8, the setting screen 200A of the display setting information 24 for the usage scene “watching” includes an action or a state name, a setting name, and a setting value as items.

The action or the state name indicates a name of the action or the state of the person.

The setting name indicates a name of a setting in association with the action or the state name. One or a plurality of setting names are associated with one action or state name.

The setting value indicates a value associated with the setting name. One setting name is associated with one setting value. However, a plurality of setting values may be associated with one setting name. A set of a setting name and a setting value may be referred to as a display setting parameter.

### <<Sleeping>>

For example, as shown in FIG. 8, setting names “location condition”, “posture condition”, “biological degree condition”, and “icon name” are associated with an action or a state name “sleeping”.

A setting value of the setting name “location condition” indicates a range of positions of the person for determining that the action of the person is “sleeping”. The range may be determined by an X section and a Y section in a map. For example, the user sets a range of a “bedroom” in the map (a floor plan of a residence) as the setting value of the setting name “location condition” associated with the action “sleeping”.

A setting value of the setting name “posture condition” indicates a posture of the person for determining that the action of the person is “sleeping”. One posture may be selected from a predetermined posture list. For example, the user selects “lying” from the predetermined posture list as the setting value of the setting name “posture condition” associated with the action “sleeping”.

A setting value of the setting name “biological degree condition” indicates the biological degree of the person and a correction amount of the biological degree for determining that the action of the person is “sleeping”. Generally, since a person who is sleeping is at rest, the vital sign value tends to be lower than usual. Here, the user may set the correction amount such that a default threshold of the vital sign degree is lowered by a predetermined amount as the setting value of the setting name “biological degree condition” associated with the action “sleeping”. Accordingly, it is possible to suppress the possibility that the terminal device 14 erroneously determines the vital sign degree as “low” even though the vital sign value is slightly lower than usual because the person is sleeping. However, it is not essential to associate the setting name “biological degree condition” with the action name “sleeping”.

A setting value of the setting name “icon name” indicates a file name of an icon used when it is determined that the action of the person is “sleeping”.

For example, when the position of the person is within the range (for example, in the bedroom) indicated by the setting value of the setting name “location condition”, the posture of the person is the posture “lying” indicated by the setting value of the setting name “posture condition”, and the vital sign degree of the person is “normal” corrected by the setting value of the setting name “biological degree condition”, the terminal device 14 determines that the action of the person is “sleeping” and displays the icon of the file name indicated by the setting value of the setting name “icon name”. Details of a display method will be described later.

### <<Watching Television>>

For example, as shown in FIG. 8, the setting names “location condition”, “posture condition”, “biological degree condition”, and “icon name” are associated with the action or the state name “watching television”.

The setting value of the setting name “location condition” indicates a range of positions of the person for determining that the action of the person is “watching television”. The range may be determined by the X section and the Y section in the map. For example, the user sets a range of a “living room” in the map (the floor plan of the residence) as the setting value of the setting name “location condition” associated with the action “watching television”.

The setting value of the setting name “posture condition” indicates a posture of the person for determining that the action of the person is “watching television”. One posture may be selected from the predetermined posture list. For example, the user selects “sitting” from the predetermined posture list as the setting value of the setting name “posture condition” associated with the action “watching television”.

A setting value of the setting name “biological degree condition” indicates the biological degree of the person and a correction amount of the biological degree for determining that the action of the person is “watching television”. For example, the person who is watching television may have a vital sign value higher than usual due to the influence of content. Here, the user may set the correction amount such that the default threshold of the vital sign degree is increased by a predetermined amount as a setting value of the setting name “biological degree condition” associated with the action “watching television”. Accordingly, it is possible to suppress the possibility that the terminal device 14 erroneously determines the vital sign degree as “high” even though the vital sign value is slightly higher than usual because the person is watching television. However, it is not essential to associate the setting name “biological degree condition” with the action name “watching television”.

The setting value of the setting name “icon name” indicates the file name of the icon used when it is determined that the action of the person is “watching television”.

For example, when the position of the person is within the range (for example, in the living room) indicated by the setting value of the “location condition”, the posture of the person is the posture “sitting” indicated by the setting value of the “posture condition”, and the vital sign degree of the person is “normal” corrected by the setting value of the setting name “biological degree condition”, the terminal device 14 determines that the action of the person is “watching television” and displays the icon of the file name indicated by the setting value of the “icon name”. Details of a display method will be described later.

### <<Abnormal State>>

For example, as shown in FIG. 8, setting names “location condition”, “posture condition”, “vital sign degree condition”, “vital sign increase and decrease change degree condition”, and “icon name” are associated with the action or the state name “abnormal state”.

The setting value of the setting name “location condition” indicates a range of positions of the person for determining that a state of the person is the “abnormal state”. In FIG. 8, an entire range of the monitoring area 8 is set as a range of the determination of the abnormal state.

A setting value of the setting name “posture condition” indicates a posture of the person for determining that the state of the person is the “abnormal state”. One posture may be selected from the predetermined posture list. For example, the user selects “lying” from the predetermined posture list as the setting value of the setting name “posture condition” associated with the state “abnormal state”.

A setting value of the setting name “vital sign degree condition” indicates a vital sign degree for determining that the state of the person is the “abnormal state”. For example, the user sets “high” and “low” as the setting value of the setting name “vital sign degree condition” associated with the state “abnormal state”.

A setting value of the setting name “vital sign increase and decrease change degree condition” indicates the degree of the vital sign increase change and the degree of the vital sign decrease change for determining that the state of the person is the “abnormal state”. For example, the user sets “rapid increase” and “rapid decrease” as the setting value of the setting name “vital sign increase and decrease change degree condition” associated with the state “abnormal state”.

The setting value of the setting name “icon name” indicates the file name of the icon used when it is determined that the state of the person is the “abnormal state”.

For example, in a case where the person is at any position in the monitoring area 8 (for example, any position in the residence), the posture of the person is the posture “lying” indicated by the setting value of the “posture condition”, the vital sign degree of the person is “high” or “low” indicated by the setting value of the “vital sign degree condition”, and the degree of the vital sign increase change or the degree of the vital sign decrease change of the person is the “rapid increase” or “rapid decrease” indicated by the setting value of the “vital sign increase and decrease change degree condition”, the terminal device 14 determines that the state of the person is the “abnormal state” and displays the icon of the file name indicated by the setting value of the setting name “icon name”. Details of a display method will be described later.

### <Biological Degree Correction Setting Information for Watching>

FIG. 9 is a diagram showing a setting screen 300A of biological degree correction setting information for the usage scene “watching” according to Embodiment 1.

The terminal device 14 displays the setting screen 300A for setting the biological degree correction setting information shown in FIG. 9 in addition to the display setting information 24 described above.

The setting screen 300A of the biological degree correction setting information includes a location name and a correction amount as items. The location name indicates the name of the location of the monitoring area 8. When the monitoring area 8 is a residence, the location name is, for example, “living room”, “toilet”, “bathroom”, or “bedroom”. The correction amount indicates a correction amount of the biological degree determination information 23 when the person is positioned at the location indicated by the location name.

For example, as shown in a first line of the biological degree correction setting information shown in FIG. 9, the user may associate the location name “toilet” with a setting in which a threshold of “high” of the vital sign degree is increased as the correction amount. In this case, the terminal device 14 determines the vital sign degree for the person positioned in the toilet in consideration of the correction amount. Accordingly, it is possible to suppress the possibility that the vital sign degree is erroneously determined as “high” even though the vital sign value is slightly higher than usual because the person is in the toilet.

For example, as shown in a second line of the biological degree correction setting information shown in FIG. 9, the user may associate the location name “bathroom” with a setting in which a threshold of “high” of the vital sign degree is increased as the correction amount. In this case, the terminal device 14 determines the vital sign degree for the person positioned in the bathroom in consideration of the correction amount. Accordingly, it is possible to suppress the possibility that the terminal device 14 erroneously determines the vital sign degree as “high” even though the vital sign value is slightly higher than usual because the person is taking a bath.

### <Monitoring Screen for Watching>

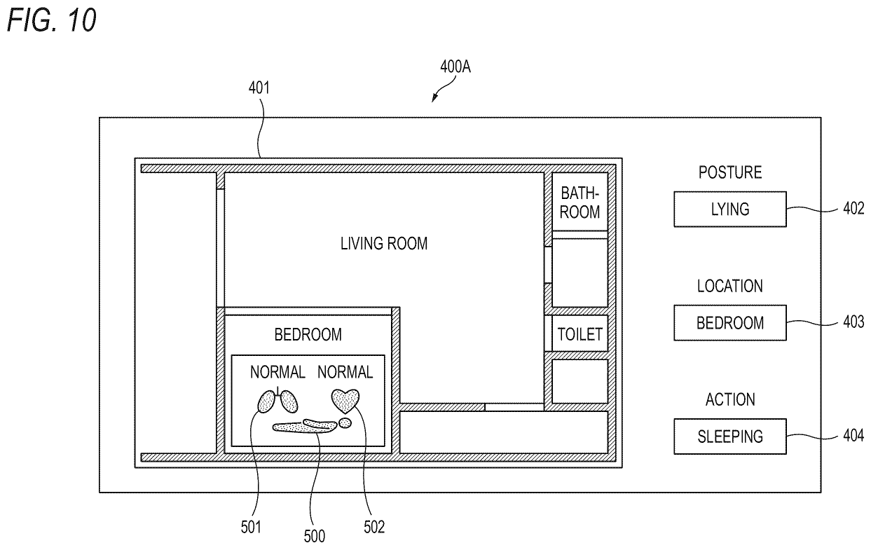

FIG. 10 is a diagram showing an example of a monitoring screen 400A when a person is sleeping in a bedroom according to Embodiment 1.

As shown in FIG. 10, the terminal device 14 displays the monitoring screen 400A. The monitoring screen 400A includes a map region 401, a posture region 402, a location region 403, and an action region 404.

The terminal device 14 displays, in the map region 401, a floor plan of the residence in the monitoring area 8 as an example of the map.

The terminal device 14 detects the posture of the person 1 based on the biological information 21 received from the biological information detection device 12, and displays the detected posture name in the posture region 402.

The terminal device 14 detects the position of the person 1 based on the biological information 21 received from the biological information detection device 12. The terminal device 14 maps the detected position of the person on the floor plan and specifies a location name of the person 1. The terminal device 14 displays the specified location name in the location region 403.

The terminal device 14 estimates the action of the person 1 based on the biological information 21 received from the biological information detection device 12, the biological degree information 22 received from the management device 13, and the display setting information 24. The terminal device 14 displays the estimated action name of the person 1 in the action region 404.

Further, the terminal device 14 displays an icon 500 corresponding to the estimated action and an icon 501 corresponding to the biological degree in the specified location of the map region 401. The icon in the present embodiment may be read as graphic information.

For example, when the posture of the person 1 estimated from the biological information 21 is “lying”, the terminal device 14 displays “lying” in the posture region 402.

For example, when the position of the person 1 estimated from the biological information 21 is “bedroom”, the terminal device 14 displays “bedroom” in the location region 403.

For example, when the action of the person 1 estimated from the display setting information 24 is “sleeping”, the terminal device 14 displays “sleeping” in the action region 404.

For example, when it is estimated that the action of the person 1 is “sleeping” based on the biological degree information 22 and the display setting information 24, the terminal device 14 displays the icon 500 of “sleeping” at the location of “bedroom” in the floor plan displayed in the map region 401.

For example, when it is determined that the degree of the respiration depth of the person 1 is “normal” and the vital sign degree of the person (for example, a heart rate degree) is “normal” based on the biological degree information 22 and the display setting information 24, the terminal device 14 displays the icon 501 indicating the degree of the respiration depth “normal” and an icon 502 indicating the vital sign (heart rate) degree “normal” in the vicinity of the icon 500.

Accordingly, the user can intuitively confirm that the person 1 who is the watching target living in the residence is currently sleeping in the bedroom and the vital sign is normal by viewing the monitoring screen 400A.

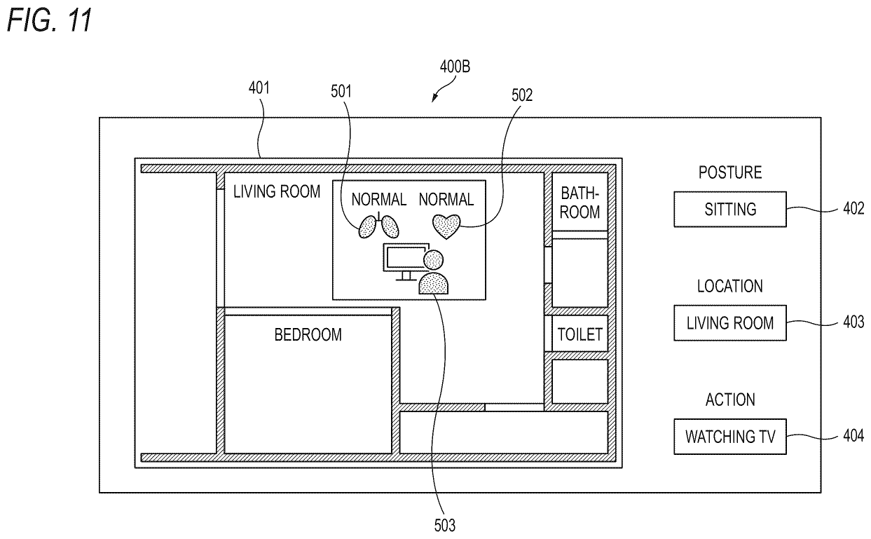

FIG. 11 is a diagram showing an example of a monitoring screen 400B when a person is watching television in a living room according to Embodiment 1.

For example, when the posture of the person 1 estimated from the biological information 21 is “sitting”, the terminal device 14 displays “sitting” in the posture region 402.

For example, when the position of the person 1 estimated from the biological information 21 is the “living room”, the terminal device 14 displays the “living room” in the location region 403.

For example, when it is estimated that the action of the person 1 is “watching television” based on the biological degree information 22 and the display setting information 24, the terminal device 14 displays “watching television” in the action region 404.

For example, when the position of the person 1 estimated from the biological information 21 is the “living room” and it is estimated from the display setting information 24 that the action of the person 1 is “watching television”, the terminal device 14 displays an icon 503 of “watching television” at the location of “living room” in the floor plan.

For example, when it is determined that the respiration depth level of the person 1 is “normal” and the vital sign degree of the person 1 (for example, the heart rate degree) is “normal” based on the biological degree information 22 and the display setting information 24, the terminal device 14 displays an icon 501 indicating the degree of the respiration depth “normal” and an icon 504 indicating the vital sign (heart rate) degree “normal” in the vicinity of the icon 503.

Accordingly, the user can intuitively confirm that the person 1 who is the watching target living in the residence is currently watching television in the living room and the vital sign is normal by viewing the monitoring screen 400B.

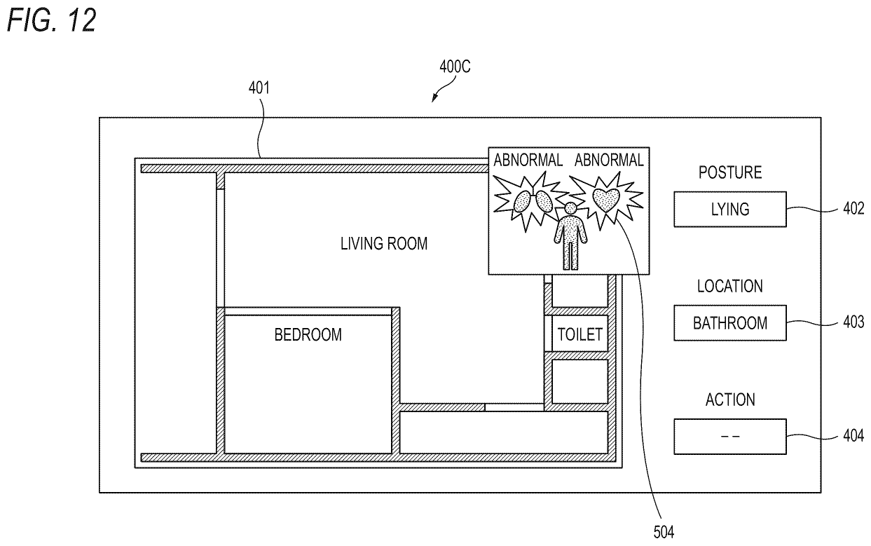

FIG. 12 is a diagram showing an example of a monitoring screen 400C when the person is in an abnormal state in a bathroom according to Embodiment 1.

For example, when the posture of the person 1 estimated from the biological information 21 is “lying”, the terminal device 14 displays “lying” in the posture region 402.

For example, when the position of the person 1 estimated from the biological information 21 is “bathroom”, the terminal device 14 displays “bathroom” in the location region 403.

For example, when it is determined that the state of the person 1 is the “abnormal state” based on the biological degree information 22 and the display setting information 24, the terminal device 14 displays the icon 504 indicating the vital sign “abnormal” at the location of the “bathroom” in the floor plan. Although the display of the action region 404 of the person 1 is omitted, alarm information such as “safety confirmation” and “emergency notification” may be displayed.

Accordingly, the user can intuitively confirm that the person who is the watching target living in the residence is currently in the bathroom and vital sign abnormality occurs by viewing the monitoring screen 400C.

### <Usage Scene: Abnormality Detection>

Next, a biological abnormality detection of an employee working in an office, which is an example of a usage scene of the detection system 10, will be described.

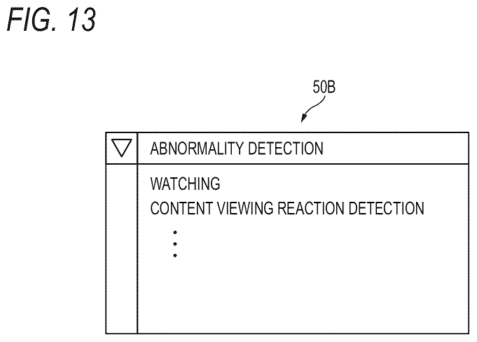

FIG. 13 is a diagram showing a selection screen 50B of a usage scene “abnormality detection” according to Embodiment 1.

In step S11 of FIG. 4, the management device 13 displays the usage scene selection screen 50B shown in FIG. 13. When the user selects “abnormality detection” as the usage scene, the management device 13 displays a setting screen 100B of the biological degree determination information 23 for the usage scene “abnormality detection” to be described below.

### <Setting Screen for Abnormality Detection>

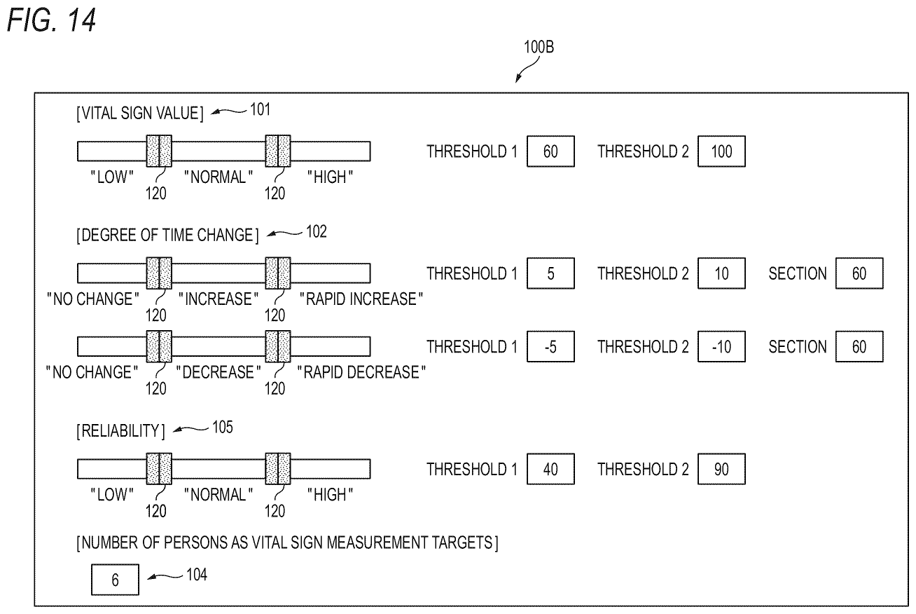

FIG. 14 is a diagram showing an example of the setting screen 100B of the biological degree determination information 23 for the usage scene “abnormality detection” according to Embodiment 1.

As shown in FIG. 14, the setting screen 100B of the biological degree determination information 23 for the usage scene “abnormality detection” includes the threshold setting region 101 of the vital sign value, the threshold setting region 102 of the degree of time change of the vital sign value, a threshold setting region 105 of the reliability, and the number-of-person-to-be-measured setting region 104.

Similarly to FIG. 7 described above, the user adjusts the threshold bars 120 of the threshold setting region 101 of the vital sign value to set thresholds of the vital sign degree. For example, since the employee who is an abnormality detection target is relatively healthy, the user may set the thresholds to be higher (that is, looser).

Similarly to FIG. 7 described above, the user adjusts the threshold bars 120 of the threshold setting region 102 of the degree of time change of the vital sign value to set thresholds of the degree of the vital sign increase change and the degree of the vital sign decrease change. For example, the user may set the thresholds to be lower (that is, stricter) since the employee who is the abnormality detection target is relatively healthy but does not want to overlook a rapid change in the vital sign.

The user adjusts the threshold bars 120 included in the threshold setting region 105 of the reliability to determine thresholds for determining the biological degree related to the height of the reliability from the reliability information included in the biological information 21. The biological degree related to the height of reliability is referred to as a reliability degree. For example, when three levels of “low”, “normal”, and “high” are set as the reliability degree, as shown in FIG. 14, the user sets the threshold 1 for distinguishing between “low” and “normal” and the threshold 2 for distinguishing between “normal” and “high”. For example, in an environment such as an office, the user may set the thresholds to be higher (that is, stricter) since an operation is hindered when erroneous detection of the abnormal state occurs frequently even though the abnormal state does not occur. The set threshold 1 and threshold 2 may be displayed as numerical values. The reliability degree is not limited to having three levels, and may have two levels or four levels or more. Each level of the reliability degree may be read as a reliability level.

Similarly to FIG. 7 described above, the user sets a maximum number of persons to be measured in the number-of-person-to-be-measured setting region 104. For example, the user may set the maximum number of persons to be measured in accordance with the number of employees working in the office.

The management device 13 generates the biological degree determination information 23 including each threshold and the maximum number of persons to be measured set by the user, and stores the biological degree determination information 23 in the storage 1003 in association with the usage scene “abnormality detection”. Accordingly, when the usage scene “abnormality detection” is selected in the monitoring processing shown in FIG. 5, the management device 13 can use the biological degree determination information 23 appropriately set for the “abnormality detection”.

### <Display Setting Information for Abnormality Detection>

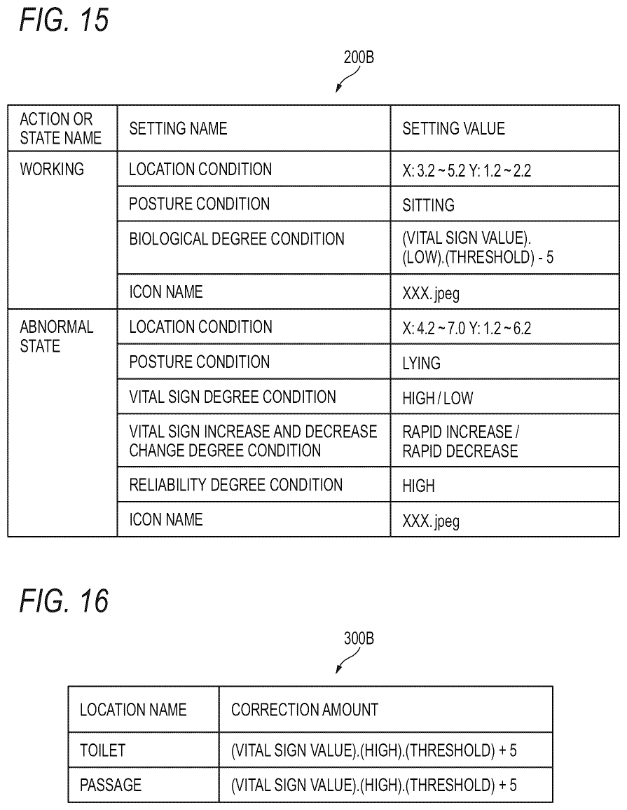

FIG. 15 is a diagram showing a setting screen of the display setting information 24 for the usage scene “abnormality detection” according to Embodiment 1.

As shown in FIG. 15, the setting screen of the display setting information 24 for the usage scene “abnormality detection” includes an action or a state name, a setting name, and a setting value as items as in FIG. 8.

### <<Working>>

For example, as shown in FIG. 15, setting names “location condition”, “posture condition”, “biological degree condition”, and “icon name” are associated with the action or the state name “working”.

A setting value of the setting name “location condition” indicates a range of positions of the person for determining that the action of the person is “working”. The range may be determined by the X section and the Y section in the map. For example, the user sets a range of a “desk” in a map (for example, a sketch of the office) as the setting value of the setting name “location condition” associated with the action “working”.

A setting value of the setting name “posture condition” indicates a posture of the person for determining that the action of the person is “working”. One posture may be selected from the predetermined posture list. For example, the user selects “sitting” from the predetermined posture list as the setting value of the setting name “posture condition” associated with the action “working”.

A setting value of the setting name “biological degree condition” indicates the biological degree of the person and a correction amount of the biological degree for determining that the action of the person is “working”. Generally, since the person who is working concentrates, the vital sign value tends to be lower than usual. Here, the user may set the correction amount such that a default threshold of the vital sign degree is lowered by a predetermined amount as the setting value of the setting name “biological degree condition” associated with the action “working”. Accordingly, it is possible to suppress the possibility that the management device 13 erroneously determines the vital sign degree as “low” even though the vital sign value is slightly lower than usual because the person is working. However, it is not essential to associate the setting name “biological degree condition” with the action name “working”.

A setting value of the setting name “icon name” indicates a file name of an icon used when it is determined that the action of the person is “working”.

When the position of the person is within the range (for example, the desk) indicated by the setting value of the “location condition”, the posture of the person is the posture “sitting” indicated by the setting value of the “posture condition”, and the vital sign degree of the person is “normal” corrected by the setting value of the setting name “biological degree condition”, the terminal device 14 determines that the action of the person is “working” and displays the icon of the file name indicated by the setting value of the setting name “icon name”. Details of a display method will be described later.

### <<Abnormal State>>

For example, as shown in FIG. 15, setting names “location condition”, “posture condition”, “vital sign degree condition”, “vital sign increase and decrease change degree condition”, “reliability degree condition”, and “icon name” are associated with the action or the state name “abnormal state”.

The setting names “location condition”, “posture condition”, “vital sign degree condition”, “vital sign increase and decrease change degree condition”, and “icon name” are similar to those in FIG. 8, and description thereof will be omitted.

A setting value of the setting name “reliability degree condition” indicates a reliability degree for determining that the state of the person 1 is the “abnormal state”. For example, the user sets “high” as the setting value of the setting name “reliability degree condition” corresponding to the state “abnormal state”. This is because, in a location such as an office where a relatively healthy person works for a large amount of time, there is a low possibility that a biological abnormality occurs as compared with an older person, and there is a possibility that erroneous detection occurs frequently at a low reliability degree and operation is hindered.

For example, when the position of the person 1 is within the range indicated by the setting value of the “location condition”, the posture of the person 1 is the posture “lying” indicated by the setting value of the “posture condition”, the vital sign degree of the person is “high” or “low” indicated by the setting value of the “vital sign degree condition”, the degree of the vital sign increase change or the degree of the vital sign decrease change of the person 1 is “rapid increase” or “rapid decrease” indicated by the setting value of the “vital sign increase and decrease change degree condition”, and the reliability degree of the person 1 is “high” indicated by the setting value of the “reliability degree condition”, the terminal device 14 determines that the state of the person 1 is the “abnormal state” and displays the icon of the file name indicated by the setting value of the setting name “icon name”. Details of a display method will be described later.

### <BiologicalDegree Correction Setting Information for Abnormality Detection>

FIG. 16 is a diagram showing a setting screen of the biological degree correction setting information for the usage scene “abnormality detection” according to Embodiment 1.

The terminal device 14 displays a setting screen 300B for setting the biological degree correction setting information shown in FIG. 16 in addition to the display setting information 24 described above.

The setting screen 300B of the biological degree correction setting information includes a location name and a correction amount as items. When the monitoring area 8 is an office, the location name may be, for example, “toilet”, “passage”, or the like.

For example, as shown in a first line of the biological degree correction setting information shown in FIG. 16, the user may associate the location name “toilet” with a setting in which a threshold of “high” of the vital sign degree is increased as the correction amount. In this case, the terminal device 14 determines the vital sign degree for the person 1 positioned in the toilet in consideration of the correction amount. Accordingly, it is possible to suppress the possibility that the vital sign degree is erroneously determined as “high” even though the vital sign value is slightly higher than usual because the person 1 is in the toilet.

For example, as shown in a second line of the biological degree correction setting information shown in FIG. 16, the user may associate the location name “passage” with a setting in which a threshold of “high” of the vital sign degree is increased as the correction amount. In this case, the terminal device 14 determines the vital sign degree of the person 1 positioned in the passage in consideration of the correction amount. Accordingly, it is possible to suppress the possibility that the terminal device 14 erroneously determines the vital sign degree as “high” even though the vital sign value is slightly higher than normal because the person is moving along the passage.

### <Monitoring Screen for Abnormality Detection>

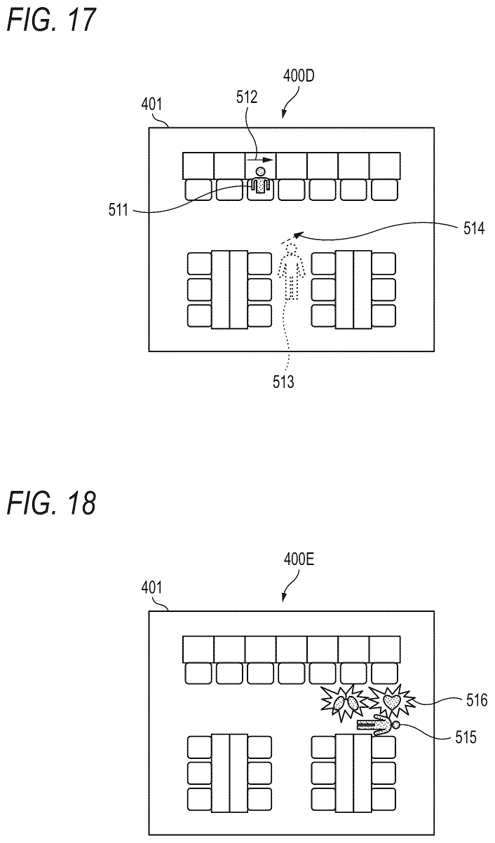

FIG. 17 is a diagram showing an example of a monitoring screen 400D when two persons are in the office according to Embodiment 1.

As shown in FIG. 17, the terminal device 14 displays the monitoring screen 400D. The monitoring screen 400D includes a map region 401.

The terminal device 14 displays, in the map region 401, the sketch of the office which is the monitoring area 8 as an example of the map.

For example, when it is estimated from the display setting information 24 that the action of the person A is “working”, the terminal device 14 displays an icon 511 of “working” at a position of a person A in the sketch displayed in the map region 401. In addition, the terminal device 14 displays the icon 511 with, for example, a solid line such that it is known that the reliability degree is “high”. In addition, the terminal device 14 displays an arrow icon (a horizontal arrow icon 512 in FIG. 17) indicating that the degree of the vital sign (heart rate) increase and decrease change is “normal” near the icon 511.

For example, when it is estimated from the display setting information 24 that the action of the person B is “moving”, the terminal device 14 displays the icon 513 indicating “moving” at the position of the person B in the sketch displayed in the map region 401. In addition, the terminal device 14 displays the icon 513 with, for example, a broken line such that it is known that the reliability degree is “low”. In addition, the terminal device 14 displays an arrow icon (an obliquely upward arrow icon 514 in FIG. 17) indicating that the degree of the vital sign (heart rate) increase and decrease change is “rapid increase” in the vicinity of the icon 513.

Accordingly, the user can intuitively confirm that the person A who is working in the office is working, the vital sign is normal, and the reliability of the vital sign is high by viewing the monitoring screen 400D. Further, the user can intuitively confirm that the person B is moving and the reliability of the vital sign is low although the vital sign change is a rapid increase by viewing the monitoring screen 400D.

FIG. 18 is a diagram showing an example of a monitoring screen 400E when there is a person in an abnormal state in the office according to Embodiment 1.

For example, when it is estimated from the display setting information 24 that the state of the person C is the “abnormal state”, the terminal device 14 displays the icon 515 indicating the “abnormal state” at the position of the person in the sketch displayed in the map region 401. The terminal device 14 displays the icon 515 with, for example, a solid line such that it is known that the reliability degree is “high”. In addition, the terminal device 14 displays an icon 516 indicating that the vital sign is “abnormal state” near the icon 515.

Accordingly, the user can intuitively confirm that the vital sign abnormality occurs in the person C who is working in the office and the reliability of the vital sign is high by viewing the monitoring screen 400E. In this case, the terminal device 14 may output an alert (for example, display of a character string related to safety confirmation or issuance of a sound) indicating that the person 1 having the biological abnormality occurs.

### Embodiment 2

In Embodiment 2, a detection system that supports installation of a sensor in a predetermined area such as a room or an office described in Embodiment 1 will be described. In Embodiment 2, the same reference numerals are given to components described in Embodiment 1, and description thereof may be omitted.

### <Usage Scene: Sensor Installation Support>

Next, sensor installation support, which is an example of a usage scene of the detection system 10, will be described.

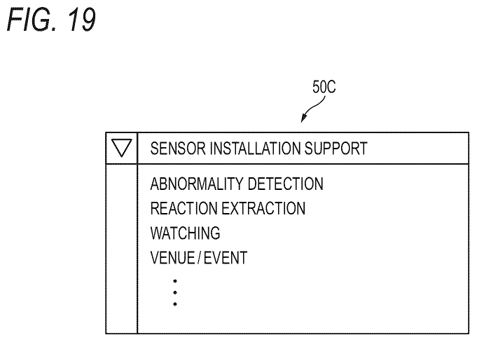

FIG. 19 is a diagram showing an example of a selection screen 50C of the usage scene “sensor installation support” according to Embodiment 2.

A user (for example, a worker who installs the sensor 11) first temporarily installs the sensor 11 in a predetermined area in which the monitoring area 8 is to be provided. Then, when the user selects the usage scene “sensor installation support” on the selection screen 50C displayed by the terminal device 14, the terminal device 14 displays a setting screen 700 of sensor installation information to be described below.

### <Setting Screen of Sensor Installation Information>

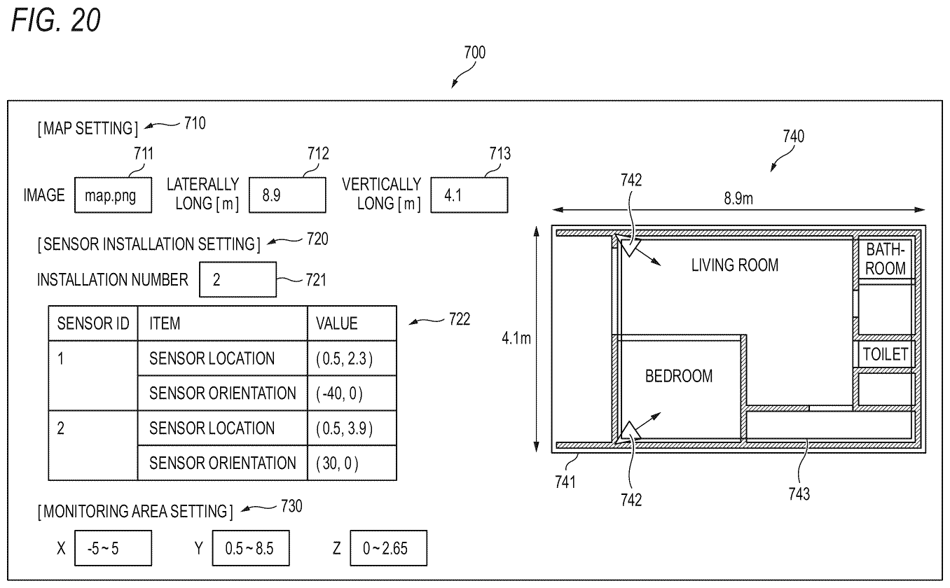

FIG. 20 is a diagram showing an example of the setting screen 700 of the sensor installation information according to Embodiment 2. The setting screen 700 of the sensor installation information is an example of support information for supporting installation of the sensor 11 in the area.

The setting screen 700 of the sensor installation information includes a map setting region 710, a sensor installation setting region 720, a monitoring area setting region 730, and a map display region 740.

The map setting region 710 includes a map image input region 711, a laterally long input region 712, and a vertically long input region 713.

In the map image input region 711, a file of a map image (for example, an image of a floor plan of a room) 741 indicating a predetermined area in which the sensor 11 is installed is input by the user.

A lateral length of the area shown in the map image 741 is input to the laterally long input region 712 by the user. A vertical length of the area shown in the map image 741 is input to the vertically long input region 713 by the user. Hereinafter, a scale of the area indicated by the values input to the laterally long input region 712 and the vertically long input region 713 is referred to as an input scale.

The sensor installation setting region 720 includes a number-of-sensors input region 721 and a sensor installation information input region 722.

The number of sensors 11 temporarily installed in the area is input to the number-of-sensors input region 721 by the user.

Sensor installation information is input to the sensor installation information input region 722 by the user. The sensor installation information includes a sensor ID for identifying the sensor 11, a value indicating an installation position of the sensor 11, and a value indicating an orientation of the sensor 11. The value indicating the installation position of the sensor 11 may include a coordinate in a horizontal direction (X direction) and a coordinate in a vertical direction (Y direction) on the map image 741. The value indicating the orientation of the sensor 11 may include an azimuth angle and a depression angle indicating the orientation of the sensor 11. However, the sensor installation information does not necessarily include both the value indicating the installation position of the sensor 11 and the value indicating the orientation of the sensor 11, and may include any one of them.

In the monitoring area setting region 730, an X section, a Y section, and a Z section indicating an XYZ space set in the monitoring area 8 in the area 11 are input by the user. The XYZ space defined by the X section, the Y section, and the Z section set here is the monitoring area 8 for the watching, the abnormality detection or the like described in Embodiment 1.

The map image 741 input to the map image input region 711 is displayed in the map display region 740. Further, in the map display region 740, a sensor image 742 indicating the position and orientation of the sensor 11 input to the sensor installation information input region 722 is superimposed on the map image 741. Accordingly, the user can easily confirm the position and orientation of the sensor 11 temporarily installed in the area by viewing the map display region 740.

In the map display region 740, the lateral length of the area input to the laterally long input region 712 and the vertical length of the area input to the vertically long input region 713 are displayed as numerical values near the map image 741.

In the map display region 740, a range image 743 indicating the range of the monitoring area 8 input to the monitoring area setting region 730 is superimposed on the map image 741.

### <Sensor Installation Confirmation Support Screen>

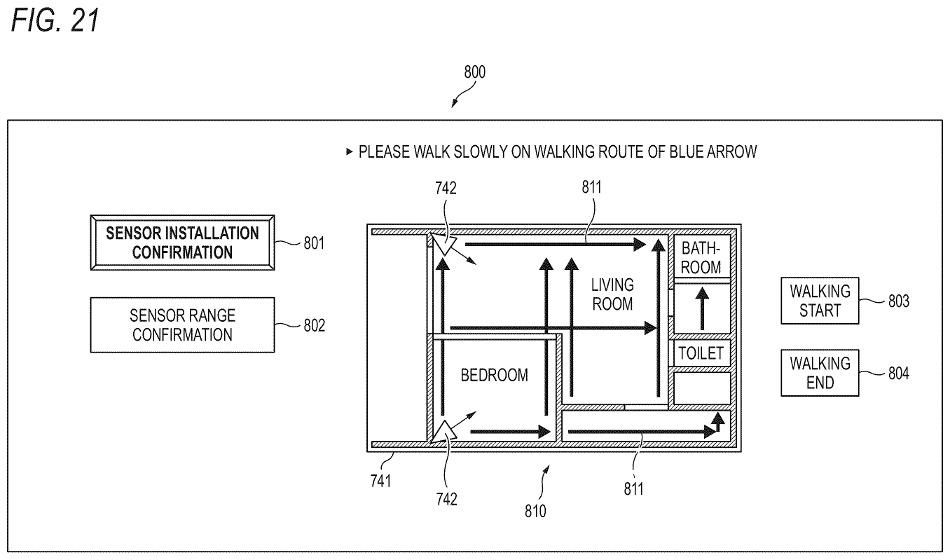

A gap may be present between the input scale input to the setting screen 700 of the sensor installation information and an actual scale of the area. Further, a deviation may be present between the position and orientation of the sensor 11 input to the setting screen 700 of the sensor installation information and the position and orientation of the sensor actually installed in the area. As shown in FIG. 21, the terminal device 14 displays a sensor installation confirmation support screen 800 that supports the correction of the deviation.

FIG. 21 is a diagram showing an example of the sensor installation confirmation support screen 800 according to Embodiment 2. The sensor installation confirmation support screen 800 is an example of the support information.

The sensor installation confirmation support screen 800 includes a sensor installation confirmation button 801, a sensor range confirmation button 802, a walking route display region 810, a walking start button 803, and a walking end button 804.

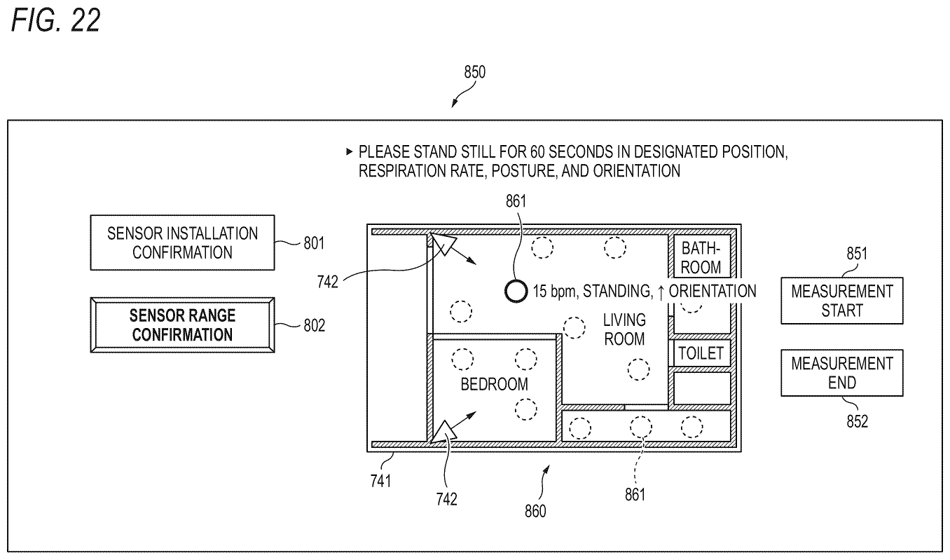

When the sensor installation confirmation button 801 is pressed, the terminal device 14 displays the sensor installation confirmation support screen 800 shown in FIG. 21. When the sensor range confirmation button 802 is pressed, the terminal device 14 displays a sensor range confirmation support screen 850 shown in FIG. 22 to be described later.

The map image 741 input to the map image input region 711 is displayed in the walking route display region 810. In the walking route display region 810, the sensor image 742 indicating the position and orientation input to the sensor installation information input region 722 is superimposed on the map image 741. In addition, in the walking route display region 810, a walking route 811 indicating a route along which the user (person) wants to walk is superimposed on the map image 741. For example, the walking route 811 is displayed by an arrow as shown in FIG. 21. In FIG. 21, a dotted arrow indicates the walking route 811 along which the user has not yet walked, and a solid arrow indicates the walking route 811 along which the user has already walked.

The terminal device 14 controls the sensor 11 to detect the movement of the position of the person in the area, thereby calculating the actual scale of the area and the actual position and orientation of the sensor 11. At this time, the terminal device 14 generates a plurality of the walking routes 811 that can calculate the actual scale of the area and the actual position and orientation of the sensor with relatively high accuracy. For example, the terminal device 14 performs image analysis on the map image 741 to estimate black straight lines each having a predetermined width as walls, and generates the plurality of walking routes 811 along the walls as much as possible. Accordingly, the terminal device 14 can accurately calculate the actual scale of the area by detecting the user moving on the walking route 811 with a sensor 11 and calculating the movement distance. In addition, the terminal device 14 can calculate the actual position and orientation of the sensor 11 by detecting the user moving on the walking route 811 with the sensor 11 and specifying the position of the user within a detectable range of the sensor 11. The generation of the plurality of walking routes 811 is not limited to this method. For example, the walking route 811 may be manually generated by the user's operation of indicating a walking start point and a walking end point in consideration of an arrangement situation of furniture or baggage in the room.

Next, an operation of the user moving along the walking route 811 and the processing of the terminal device 14 at that time will be described. For example, the user and the terminal device 14 perform the operation and the processing of steps S101 to S108 (not shown) to be described below.

(S101) The user moves to a starting point of the walking route 811 indicated by the dotted arrow while viewing the walking route display region 810 displayed on the terminal device 14, and presses the walking start button 803. When the walking start button 803 is pressed, the terminal device 14 controls the sensor 11 to start detecting the biological information 21 of the user.

(S102) The user moves along the walking route 811 indicated by the dotted arrow.

At this time, the sensor 11 measures the position, speed, vital sign value, or the like of the moving user at any time, and generates the biological information 21.

(S103) The user moves to an end point of the walking route 811 of the dotted arrow and presses the walking end button 804. When the walking end button 804 is pressed, the terminal device 14 controls the sensor 11 to end the detection.

(S104) The terminal device 14 changes the walking route 811 indicated by the dotted arrow that has been detected to the walking route 811 indicated by the solid line arrow.

(S105) The user and the terminal device 14 also perform the operation and the processing of step S101 to step S104 described above for the other walking routes 811 indicated by the dotted arrows.

(S107) After all the walking routes 811 indicated by the dotted arrows are changed to the walking routes 811 indicated by the solid line arrows, the terminal device 14 calculates the actual scale of the area and the actual position and orientation of the sensor 11 based on the biological information 21 acquired from the biological information detection device 12.

(S108) The terminal device 14 corrects the deviation of the input scale based on the calculated actual scale of the area. Further, the terminal device 14 corrects the deviation of the position and the orientation of the sensor 11 input to the sensor installation information input region 722 based on the calculated actual position and orientation of the sensor 11. The terminal device 14 stores the corrected scale of the area and the position and orientation of the sensor 11 in the storage 1003 (see FIG. 24).

Through the above processing, the terminal device 14 can set a more accurate scale of the area and a more accurate position and orientation of the sensor 11.

### <Sensor Range Confirmation Support Screen>

When a range where sensing of a person is not possible is present in the monitoring area 8, the watching, the abnormality detection, or the like described in Embodiment 1 cannot be realized with high reliability. As shown in FIG. 22, the terminal device 14 displays the sensor range confirmation support screen 850 that supports confirmation of a range where sensing of a person is possible and/or the range in which sensing of a person is not possible.

FIG. 22 is a diagram showing an example of the sensor range confirmation support screen 850 according to Embodiment 2. The sensor range confirmation support screen 850 is an example of the support information.

The sensor range confirmation support screen 850 includes a sensor installation confirmation button 801, a sensor range confirmation button 802, a measurement point display region 860, a measurement start button 851, and a measurement end button 852.

When the sensor installation confirmation button 801 is pressed, the terminal device 14 displays the sensor installation confirmation support screen 800 shown in FIG. 21. When the sensor range confirmation button 802 is pressed, the terminal device 14 displays the sensor range confirmation support screen 850 shown in FIG. 22.

The map image 741 input to the map image input region 711 is displayed in the measurement point display region 860. In the measurement point display region 860, the sensor image 742 indicating the corrected position and orientation of the sensor 11 through the sensor installation confirmation support screen 800 is superimposed on the map image 741. In addition, in the measurement point display region 860, a measurement point 861 indicating a point at which the user wants to be positioned is superimposed on the map image 741. For example, as shown in FIG. 22, the measurement point 861 is displayed as a circle. In FIG. 22, a dotted circle indicates the measurement point 861 where the user has not yet been measured, and a solid line circle indicates the measurement point 861 where the user has already been measured.

For example, the terminal device 14 analyzes the map image 741 of the sketch, and distributes and arranges the plurality of measurement points 861 in the monitoring area 8. For example, the terminal device 14 estimates black straight lines each having a predetermined width in the map image 741 as walls, and arranges the measurement points 861 near the center and the corner of the room surrounded by the walls. Accordingly, the terminal device 14 can determine whether a range of a predetermined size around the measurement point 861 is a range in which sensing can be performed by measuring the user positioned at the measurement point 861 with the sensor 11. The generation of the plurality of measurement points 861 is not limited to this method. For example, the measurement point 861 may be manually generated by a user's operation of indicating a position to be a location of stay in consideration of an arrangement situation of furniture or baggage in the room.

Next, an operation of the user moving to the measurement point 861 and the processing of the terminal device 14 at that time will be described. For example, the user and the terminal device 14 perform operation and processing of steps S201 to S208 to be described below.

(S201) The user moves to the measurement point 861 indicated by the dotted circle while viewing the measurement point display region 860 displayed on the terminal device 14, and presses the measurement start button 851. When the measurement start button 851 is pressed, the terminal device 14 displays a respiration rate, a posture, and an orientation that the user is desired to achieve at the measurement point 861, and a time (hereinafter, referred to as a rest time) during which the user is desired to stand still at the measurement point 861. Then, the terminal device 14 controls the sensor 11 to start measuring the vital sign value or the like of the user.

(S203) The user stands still at the measurement point 861 for the displayed rest time with the displayed respiration rate, posture, and orientation. The sensor 11 measures the vital sign value of the user standing still at the measurement point 861 at any time, and generates the biological information 21.

(S204) After the displayed rest time has elapsed, the user presses the measurement end button 852. When the measurement end button 852 is pressed, the terminal device 14 controls the sensor 11 to end the measurement.

(S205) The terminal device 14 changes the measurement point 861 indicated by the dotted circle where the measurement is performed to the measurement point 861 indicated by the solid line circle.

(S206) The user and the terminal device 14 perform the operation and the processing of step S201 to step S205 described above for the other dotted circles.

(S207) After all the dotted circles are changed to the solid line circles, the terminal device 14 specifies the measurement point 861 at which the user can be detected and the measurement point 861 at which the user cannot be detected based on the biological information 21 acquired from the biological information detection device 12. The terminal device 14 specifies the measurement point 861 at which the vital sign value can be measured and the measurement point 861 at which the vital sign value cannot be measured based on the biological information 21 acquired from the biological information detection device 12.

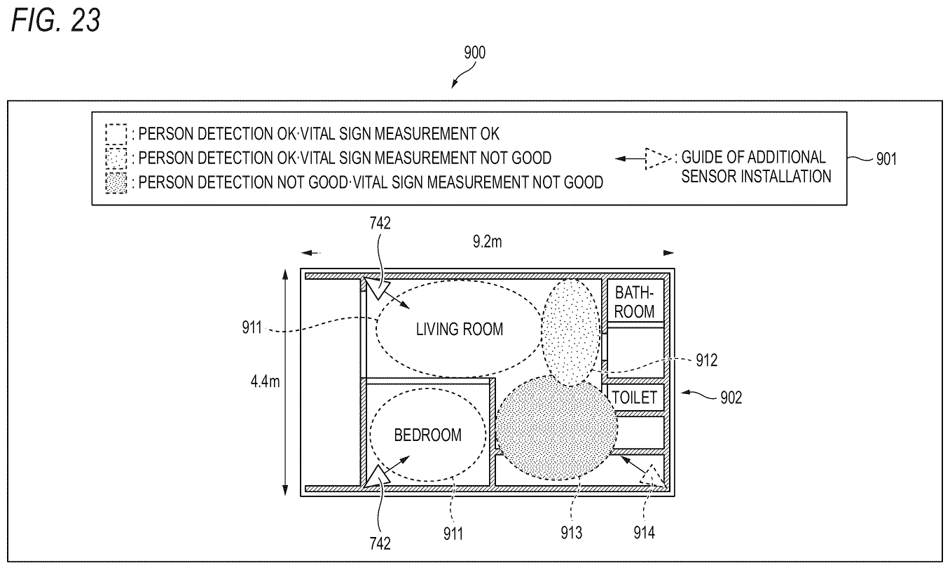

(S208) The terminal device 14 specifies both-detectable ranges 911 (see FIG. 23), a person detectable range 912 (see FIG. 23), and an undetectable range 913 (see FIG. 23) based on results of the detection and the measurement at the measurement points 861 in step S207. The both-detectable ranges 911 are ranges in which the presence of a person is detectable and the vital sign value indicating the activity state of the person is measurable. The person detectable range 912 is a range in which the presence of a person is detectable and the vital sign value indicating the activity state of the person is unmeasurable. The undetectable range 913 is a range in which the presence of a person is undetectable and the vital sign value indicating the activity state of the person is unmeasurable.

### <Result Confirmation Screen>

The terminal device 14 displays results obtained through the sensor installation confirmation support screen 800 and the sensor range confirmation support screen 850 as a result confirmation screen 900 shown in FIG. 23.

FIG. 23 is a diagram showing an example of the result confirmation screen 900 according to Embodiment 2. The result confirmation screen 900 is an example of the support information.

The result confirmation screen 900 includes a description region 901 and a result display region 902.

In the description region 901, description of how to see the result displayed in the result display region 902 is displayed.

The map image 741 and a scale of the corrected area are displayed in the result display region 902. In addition, in the result display region 902, a sensor image 742 indicating the corrected position and orientation of the sensor 11 is superimposed on the map image 741. Accordingly, the user can check the more accurate area scale and the more accurate position and orientation of the sensor 11 by viewing the result display region 902.

In addition, in the result display region 902, when the both-detectable ranges 911, the person detectable range 912, and the undetectable range 913 specified in step S208 described above are present, respectively, they are superimposed and displayed on the map image 741. Accordingly, the user can check the both-detectable ranges 911, the person detectable range 912, and the undetectable range 913 in the monitoring area 8 by viewing the result display region 902. Thus, the user can easily analyze how to adjust the position and orientation of the sensor 11 installed in the area so that the person detectable range 912 or the undetectable range 913 in the monitoring area 8 can be set as the both-detectable ranges 911.

Further, when the person detectable range 912 or the undetectable range 913 is present, the terminal device 14 may calculate at which position and in which orientation the additional sensor 11 should be installed in order to set these ranges as the both-detectable ranges 911. In the calculation of the installation position and orientation, the optimal additional sensor 11 can be installed by using specifications of various sensors, information of the detection range by the existing sensor, or the like. Then, the terminal device 14 may superimpose and display the additional sensor image 914 indicating the calculated position and orientation of the additional sensor 11 on the map image 741. The additional sensor image 914 may be read as information recommending addition of the new sensor 11. Accordingly, the user can easily analyze, by viewing the result display region 902, at which position and in which orientation the additional sensor 11 is installed, the person detectable range 912 or the undetectable range 913 in the monitoring area 8 can be set as the both-detectable ranges 911.

### <Hardware Configuration>

Although the embodiments according to the present disclosure have been described in detail with reference to the drawings, functions of the biological information detection device 12, the management device 13, and the terminal device 14 described above can be implemented by a computer program.

FIG. 24 is a diagram showing a hardware configuration of a computer that implements the functions of each device according to the present disclosure by the computer program.

A computer 1000 includes a processor 1001, a memory 1002, a storage 1003, an input device 1004, an output device 1005, a communication device 1006, a graphics processing unit (GPU) 1007, a reading device 1008, and a bus 1009.

The processor 1001, the memory 1002, the storage 1003, the input device 1004, the output device 1005, the communication device 1006, the GPU 1007, and the reading device 1008 are connected to the bus 1009 and can transmit and receive data in both directions via the bus 1009.

The processor 1001 is a device that executes a computer program stored in the memory 1002 to implement the function blocks described above. Examples of the processor 1001 include a central processing unit (CPU), a micro processing unit (MPU), a controller, a large scale integration (LSI), an application specific integrated circuit (ASIC), a programmable logic device (PLD), and a field-programmable gate array (FPGA).

The memory 1002 is a device that is implemented by a volatile storage medium, and that stores a computer program and data handled by the computer 1000. However, at least a part of the memory 1002 may be implemented by a nonvolatile storage medium.

The storage 1003 is a device that is implemented by a nonvolatile storage medium, and that stores a computer program and data handled by the computer 1000. Examples of the storage 1003 include a hard disk drive (HDD) or a solid state drive (SSD).

The input device 1004 is a device that receives data to be input to the processor 1001. Examples of the input device 1004 include a keyboard, a mouse, a touch pad, or a microphone.

The output device 1005 is a device that outputs data generated by the processor 1001. Examples of the output device 1005 include a display or a speaker.

The communication device 1006 is a device that transmits and receives data to and from another device via a communication network. The communication device 1006 may include a transmitter that transmits data and a receiver that receives data. The communication device 1006 may support either wired communication or wireless communication. Examples of the wired communication include Ethernet (registered trademark). Examples of the wireless communication include IEEE 802.11, Wi-Fi (registered trademark), Bluetooth (registered trademark), LTE, 4G, and 5G.

The GPU 1007 is a device that processes image depiction at high speed. The GPU 1007 may be used for processing (for example, deep learning) of artificial intelligence (AI).

The reading device 1008 is a device that reads data from a recording medium such as a digital versatile disk read only memory (DVD-ROM) or a universal serial bus (USB) memory.

The content of the present disclosure can be expressed as follows.

### <A-1>

A detection system (10) includes:
at least one radar type sensor (11) installed in a predetermined area;
a biological information detection device (12) configured to detect biological information (21) of a person (1) in the area based on sensor data output from the sensor; and
a terminal device (14) configured to generate and display support information (for example, a setting screen 700, a sensor installation confirmation support screen 800, or a sensor range confirmation support screen 850, and a result confirmation screen 900 of sensor installation information) for supporting the installation of the sensor using the detected biological information.

Accordingly, the terminal device can display the support information for supporting the installation of the sensor in the area using the biological information detected for the person in the area. Accordingly, the user can appropriately install the sensor in the area by viewing the displayed support information.

### <A-2>

In the detection system according to A-1, the terminal device corrects information indicating an installation position or orientation of the sensor in the area input by a user using the detected biological information, and displays the support information including the corrected information indicating the installation position or orientation of the sensor.

Accordingly, the terminal device can correct the information indicating the installation position or orientation of the sensor input by the user and display the information indicating the installation position or orientation of the sensor with higher accuracy.

### <A-3>

In the detection system according to A-1 or A-2, the terminal device displays a map image (741) corresponding to the area and a walking route (811) superimposed on the map image, and instructs the person to move along the walking route.

Accordingly, the person can move along the walking route instructed from the terminal device.

### <A-4>

In the detection system according to A-3,
the terminal device corrects information indicating a scale of the area input by a user using the biological information detected for the person moving along the walking route, and displays the support information including the corrected information indicating the scale of the area.

Accordingly, the terminal device can correct the information indicating the scale of the area input by the user and display the corrected information indicating the scale of the area with higher accuracy.

### <A-5>

In the detection system according to any one of A-1 to A-4,
the terminal device displays a map image corresponding to the area and a measurement point superimposed on the map image, and instructs the person to be positioned at the measurement point.

Accordingly, the person can be positioned at the measurement point instructed from the terminal device.

### <A-6>

In the detection system according to A-5,
the terminal device specifies a both-detectable range, which is a range in which presence of the person is detectable and an activity state of the person is measurable by the sensor in the area, using the biological information detected for the person positioned at the measurement point, and displays the support information including the both-detectable range.

In this way, the terminal device can cause the user to recognize the both-detectable range in the area.

### <A-7>

In the detection system according to A-5 or A-6,
the terminal device specifies a person detectable range, which is a range in which presence of the person is detectable and an activity state of the person is unmeasurable by the sensor in the area, using the biological information detected for the person positioned at the measurement point, and displays the support information including the person detectable range.

Accordingly, the terminal device can cause the user to recognize the person detectable range in the area.

### <A-8>

In the detection system according to any one of A-5 to A-7,
the terminal device specifies an undetectable range, which is a range in which an activity state of the person is unmeasurable by the sensor in the area, using the biological information detected for the person positioned at the measurement point, and displays the support information including the undetectable range.

Accordingly, the terminal device can cause the user to recognize the undetectable range in the area.

### <A-9>

In the detection system according to A-8,
the terminal device displays the support information including information recommending addition of a new sensor based on the undetectable range.

Accordingly, the terminal device can recommend the user to add the new sensor for eliminating the undetectable range.

### <A-10>

A detection method includes:
detecting biological information (21) of a person (1) in a predetermined area based on sensor data output from at least one radar type sensor (11) installed in the area; and
generating and displaying support information (for example, a setting screen 700, a sensor installation confirmation support screen 800, or a sensor range confirmation support screen 850, and a result confirmation screen 900 of sensor installation information) for supporting installation of the sensor using the detected biological information.

By performing the above detection method, it is possible to display the support information for supporting the installation of the sensor in the area on, for example, a terminal device using biological information detected for the person in the area. Accordingly, the user can appropriately install the sensor in the area by viewing the displayed support information.

### <A-11>

A non-transitory computer-readable storage medium having a computer program stored thereon and readable by a computer, the computer program, when executed by the computer, causing the computer to perform:
detecting biological information (21) of a person (1) in a predetermined area based on sensor data output from at least one radar type sensor (11) installed in the area;
and generating and displaying support information (for example, a setting screen 700, a sensor installation confirmation support screen 800, or a sensor range confirmation support screen 850, and a result confirmation screen 900 of sensor installation information) for supporting installation of the sensor using the detected biological information.

Accordingly, the computer that executes the detection program can display the support information for supporting the installation of the sensor in the area using the biological information detected for the person in the area. Accordingly, the user can appropriately install the sensor in the area by viewing the displayed support information.

Although the embodiments have been described above with reference to the accompanying drawings, the present disclosure is not limited thereto. It is apparent to those skilled in the art that various modifications, corrections, substitutions, additions, deletions, and equivalents can be conceived within the scope described in the claims, and it is understood that such modifications, corrections, substitutions, additions, deletions, and equivalents also fall within the technical scope of the present disclosure. In addition, components in the embodiments described above may be combined freely in a range without departing from the gist of the invention.

### INDUSTRIAL APPLICABILITY

The technique of the present disclosure is useful for a system, a method, a program, or the like for detecting a biological state of a person using a radar type sensor.

What is claimed is:

1. A detection system comprising:
at least one radar type sensor installed in a predetermined area;
a biological information detection device configured to detect biological information of a person in the area based on sensor data output from the sensor; and
a terminal device configured to generate and display support information for supporting installation of the sensor using the detected biological information.

2. The detection system according to claim 1, wherein
the terminal device corrects information indicating an installation position or orientation of the sensor in the area input by a user using the detected biological information, and displays the support information including the corrected information indicating the installation position or orientation of the sensor.

3. The detection system according to claim 1, wherein
the terminal device displays a map image corresponding to the area and a walking route superimposed on the map image, and instructs the person to move along the walking route.

4. The detection system according to claim 3, wherein
the terminal device corrects information indicating a scale of the area input by a user using the biological information detected for the person moving along the walking route, and displays the support information including the corrected information indicating the scale of the area.

5. The detection system according to claim 1, wherein
the terminal device displays a map image corresponding to the area and a measurement point superimposed on the map image, and instructs the person to be positioned at the measurement point.

6. The detection system according to claim 5, wherein
the terminal device specifies a both-detectable range, which is a range in which presence of the person is detectable and an activity state of the person is measurable by the sensor in the area, using the biological information detected for the person positioned at the measurement point, and displays the support information including the both-detectable range.

7. The detection system according to claim 5, wherein
the terminal device specifies a person detectable range, which is a range in which presence of the person is detectable and an activity state of the person is unmeasurable by the sensor in the area, using the biological information detected for the person positioned at the measurement point, and displays the support information including the person detectable range.

8. The detection system according to claim 5, wherein
the terminal device specifies an undetectable range, which is a range in which an activity state of the person is unmeasurable by the sensor in the area, using the biological information detected for the person positioned at the measurement point, and displays the support information including the undetectable range.

9. The detection system according to claim 8, wherein
the terminal device displays the support information including information recommending addition of a new sensor based on the undetectable range.

10. A detection method comprising:
detecting biological information of a person in a predetermined area based on sensor data output from at least one radar type sensor installed in the area; and
generating and displaying support information for supporting the installation of the sensor using the detected biological information.

11. A non-transitory computer-readable storage medium having a computer program stored thereon and readable by a computer, the computer program, when executed by the computer, causing the computer to perform:
detecting biological information of a person in a predetermined area based on sensor data output from at least one radar type sensor installed in the area; and
generating and displaying support information for supporting the installation of the sensor using the detected biological information.
